(19) United States
(12) Patent Application Publication
(10) Pub. No.: US 2014/0348774 A1
(43) Pub. Date: Nov. 27, 2014

(54) METHOD FOR TREATING WOUNDS AND TISSUE REPAIR

(71) Applicants: Michael Strobel, Northfield, MN (US); Gene Komer, Northfield, MN (US)

(72) Inventors: Michael Strobel, Northfield, MN (US); Gene Komer, Northfield, MN (US)

(73) Assignee: Aurora Pharmaceutical, LLC, Northfield, MN (US)

(21) Appl. No.: 14/299,478

(22) Filed: Jun. 9, 2014

Related U.S. Application Data
(63) Continuation-in-part of application No. 13/316,186, filed on Dec. 9, 2011, now abandoned.
(60) Provisional application No. 61/421,999, filed on Dec. 10, 2010.

(51) Int. Cl. A61K 31/79; A61K 31/5375; A61K 31/215; A61L 26/00; A61K 31/167; A61K 31/235; A61K 31/7016; A61K 31/4166; A61K 31/085; A61K 36/899

(52) U.S. Cl. CPC A61K 31/79; A61K 31/085; A61K 31/5375; A61K 31/215; A61K 36/899; A61K 31/167; A61K 31/235; A61K 31/7016; A61K 31/4166; A61L 26/0066; A61L 26/0014; A61L 26/0019; A61L 2300/404; A61L 2300/202
USPC 424/78.06; 514/718; 514/239.5; 514/531; 424/750; 514/620; 514/53; 514/369

(57) ABSTRACT

The present inventions relate to a number of fields including wound healing, a waterless antibacterial disinfectant, a surgical scrub, insect repellant, and methods of treating traumatic injuries, particularly those on the battlefield where there is a need to stabilize the patient and prevent infection.

### CROSS-REFERENCE TO RELATED APPLICATIONS

This application is a continuation-in-part of U.S. patent application Ser. No. 13/316,186 filed on Dec. 9, 2011, which claims priority to U.S. Provisional Application No. 61/421,999, filed on Dec. 10, 2010, the disclosures of which are incorporated in their entireties herein by reference.

### FIELD OF THE INVENTION

The present inventions relate to a number of fields including wound healing, a waterless antibacterial disinfectant, a surgical scrub, skin infections, insect treatment and/or repellant for wounds, and methods of treating surgical incisions, both pre and post surgically, to traumatic injuries, particularly those on the battlefield where there is a need to stabilize the patient and prevent infection.

### BACKGROUND OF THE INVENTION

The inventions relate to methods for wound healing and tissue repair by promoting the growth of skin tissue with which they are in contact. The invention involves treating the injured tissue with biocompatible barriers which protect the injured tissue from infection by microbial organisms.

Early materials used to treat skin wounds included medicated and unmedicated cotton wools, gauzes, tows, and lints; gauze and cotton tissues, bandages, jaconet, oiled silk and emplastrums. From 1960 on, a new generation of products was developed based on the realization that the control of micro-environments was necessary if wound healing was to progress to the optimum degree.

Advances in the development of synthetic polymers produced the most radical changes in wound care dressings as factors such as water vapor, oxygen permeability, bacterial impermeability, and selective absorption could be incorporated into new formulations along with specific requirements such as conformability, non-adherence, and adhesiveness. This family of polymeric products included polymeric foams, polymeric films, particulate and fibrous polymers, hydrogels and hydrocolloids.

A common problem in the management of both acute and chronic wounds is the maintenance of an optimal level of moisture over the wound bed during heavy exudate drainage. This is usually, but not always, an early stage of healing. Most moist wound dressing technologies such as thin films, hydrocolloid dressings and hydrogels are typically overwhelmed by the accumulated exudate moisture during this heavy drainage phase. Management of moisture during heavy exudate drainage often necessitates the use of gauze or sponge packings that wick away excess moisture from the wound bed, thin film coverings that trap exudate fluid over the wound bed, or calcium alginate dressings that chemically bind exudate moisture due to the hydroscopic properties of the seaweed extract.

Known hydrocolloid dressings are subject to a number of drawbacks. The major disadvantages of these dressings include the potential to disintegrate in the presence of excess fluid at the wound site, and minimal, virtually negligible, control over water loss from the wound. This latter disadvantage is particularly important, as excess water loss from a wound will cause an increase in heat loss from the body as a whole, potentially leading to hypermetabolism. In addition, hydrocolloid dressings require frequent dressing changes.

There has also been proposed the use of a biocompatible wound dressing based on fibrin. One mechanism for hemostasis, i.e., prevention of blood loss, of a mammal is the formation of a blood clot. Clot formation in humans, i.e., blood coagulation, occurs by means of a complex cascade of reactions with the final steps being the conversion of fibrinogen—-a monomer—by thrombin, calcium ions and activated factor XIII to form ultimately cross linked fibrin II polymer, which is the fibrin clot.

Wound dressings have also been combined with a biodegradable carrier material. Common carriers include natural or chemically modified collagen, keratin, gelatin, carbohydrates or cellulose derivatives. Synthetic, biodegradable polymer carriers have also been proposed. These include polyhydroxycarboxylic acids, polyesters, polycyanoacrylates, polyamino acids, polyalcohols and silicones. These carrier materials are commonly employed as a web or as a fabric.

Collagen carriers suffer from numerous deficiencies. Collagen films do not readily conform to varied wound shapes. Furthermore, some collagen wound dressings have poor fluid absorption properties and undesirably enhance the pooling of wound fluids.

Wound dressings have also been combined with numerous pharmacological and/or antibiotic compositions. Examples of such compositions include, but not are not limited to, antifungal compositions, anti-viral compositions, antibacterial compositions, and antiparasitic compositions. Examples of antimicrobial compositions that can be used in the present invention include, but are not limited to, isoniazid, ethambutol, clofazimine, rifabutin, fluoroquinolones, pyrazinamide, streptomycin, ofloxacin, ganciclovir, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, erythromycin, ciprofloxacin, clindamycin, lincomycin, acyclovir, trifluorouridine, pentamidine, atovaquone, paromomycin, diclazaril, acyclovir, trifluorouridine, foscarnet, penicillin, gentamicin and sparfloxacin.

On the veterinary non-human side, each year in the United States, over 15 million castration procedures are performed on male calves destined for beef production. Although the benefits of castration are accepted in most countries, all the methods of castration result in behavioral changes indicative of pain and distress. Calves also experience discomfort during dehorning and branding. Thus, the industry needs a single product that is safe and effective in reducing pain and the risk of infection, resulting in a net benefit to the welfare of the animal.

Again, on the veterinary non-human side, each year in the United States, over 100 million pigs undergo a battery of processing procedures during the first few days or weeks of their life. These include tail docking, castration, tagging and ear notching. Each of these procedures involves a degree of tissue damage resulting in the pig experiencing pain. The pain pigs experience from these procedures may result in reduced performance The industry needs a single product that is safe and effective in reducing pain and the risk of infection, resulting in a net benefit to the welfare of the animal.

Human and non-human body tissues are oftentimes subjected to undesirable afflictions such as irritation, decay or damage of bone or soft tissue. Irritation can be reflected in inflammation, decay can involve erosion and/or decomposition of tissue, and damage can be a wound or fracture. The present invention involves topically treating mammalian, preferably human and domestic animal, tissue with a flexible wound dressing containing certain coating materials to decrease the impact of such afflictions. The device of the instant invention is a flexible thin film containing a topical organic iodine complex alcohol solution which, when applied to the skin, forms a durable barrier which lasts for 24 hours or more and provides antibacterial, antifungal, and antiviral activity.

### SUMMARY OF THE INVENTION

The present invention encompasses methods for treating injured tissues in both humans and non-human animals The present invention contemplates flexible barriers that keeps injured tissues clean and protect the injured tissues from potential infections through its disinfectant activity.

The present invention may be applied to injured tissue by any method known in the art, including, but not limited to, as a liquid, spray, or thin film.

The present invention encompasses methods for treating routine surgical incisions, both pre- and post-op.

Methods of the present invention may also include topical applications to companion animals to prevent chewing and licking of superficial wounds and surgical incisions.

Methods contemplated by the present invention may be used in the treatment of wounds incurred during the castration and tail docking in sheep, pigs, and dogs and well as dehorning wounds in cattle.

Methods of the present invention may also be used as a topical treatment to prevent infection and navel sucking in calves, foals, lambs, and kids.

Methods of the present invention may comprise agents which enhance the flexibility of the composition on the skin. Such agents include, but are not limited to glycerine and propylene glycol. Said agents may be added to the compositions in amounts of 0.1-5%.

Additional therapeutic compositions that promote the wound healing process may be incorporated into the methods of the present invention. For example, methods may include antimicrobial compositions, including but not limited to antifungal compositions, antibacterial compositions, antiviral compositions and antiparasitic compositions.

One embodiment of the invention encompasses a process for enhancing the normal healing processes of a wound by providing at the area of a wound, a flexible wound dressing comprising a topical organic iodine complex alcohol solution which, when applied to the skin, forms a durable barrier which lasts for 24 hours or more and provides antibacterial, antifungal and antiviral activity.

Another embodiment of the invention encompasses a method for using a Povidone Iodine solution containing 0.05 to 5.0 percent titratable iodine with a preferred concentration of 2.0 percent titratable iodine that provides a flexible wound dressing.

Another embodiment of the invention encompasses a process for enhancing the normal healing processes of a wound by providing at the area of a wound a flexible wound dressing comprising topical Nonylphenoxy-polyethyoxyethanol-iodine complex (CAS 35860-86-7), which, when applied to the skin, forms a durable barrier which lasts for 24 hours or more and provides antibacterial, antifungal and antiviral activity.

Another embodiment of the invention encompasses a method for the use of a Nonylphenoxy-polyethyoxyethanol-iodine complex solution containing 0.05 to 5.0 percent titratable iodine with a preferred concentration of 2.0 percent titratable iodine that provides a flexible wound dressing.

Another embodiment of the invention encompasses a process for enhancing the normal healing processes of a wound by providing at the area of a wound, a flexible wound dressing comprising a mixture of the two iodine complexes is used with a range of 0.05 to 5.0 percent titratable iodine from PVPI and a range of 5.0 to 0.05 percent titratable iodine, respectively, from nonylphenoxypolyethyoxyethanol-iodine complex with a preferred concentration of 2.0 percent titratable iodine total from both sources.

Another embodiment of the invention encompasses a process of using compositions in accordance with the present invention for enhancing the normal healing processes of a wound by providing at the area of a wound, a topical anesthetic to be used for rapid pain relief with a use range of 0.01 to 5.0 percent. Examples of a topical anesthetic with those familiar in the art is the use of lidocaine and its salts with a preferred concentration of 1 to 2 percent. Other topical anesthetics within the purview of the present invention include, but are not limited to, benzocaine, butamben, dibucaine, menthol, phenol, oxybuprocaine, pramoxine, proparacaine, proxymetacaine, and tetracaine (also named amethocaine).

Another embodiment of the invention encompasses a process of using compositions in accordance with the present invention for enhancing the normal healing processes of a wound by providing at the area of a wound an analgesic to be used for pain relief. Examples of analgesics include, but are not limited to, paracetamol, acetaminophen, camphor, capsicum, menthol, the non-steroidal anti-inflammatory drugs (NSAIDs), and opioid drugs such as morphine and opium.

Another embodiment of the invention encompasses a process for enhancing the normal healing processes of a wound by providing at the area of a wound, a topical anesthetic of pramoxine HCl which provides for a longer duration of pain relief and which provides antipruritic activity which is important for treating lick granulomas in canine species. The pramoxine HCl use range is 0 to 2 percent with a preferred concentration of 1 percent.

Another embodiment of the invention encompasses the use of natural and artificial permethrin as an insect repellent with a preferred claim to prevent flies from laying eggs at or in a wound site.

Another embodiment of the invention encompasses a process of using compositions in accordance with the present invention for enhancing the normal healing processes of insect bite wounds by providing at the area of a wound lidocaine, pramoxine, permethrin, or any of their salts, with a preferred concentration of 1 to 2 percent.

Another embodiment of the invention encompasses the use of bitrex, lidocaine, and/or pramoxine may be used in treating or preventing skin infections.

Another embodiment of the invention encompasses the use of citronella and/or its component parts as an insect repellent, a bittering agent and an olfactory repellent.

Another embodiment of the invention encompasses the use of denatonium benzoate as a bittering agent to prevent chewing and biting wound sites such as encountered in castration and docking of tails in young porcine.

Another embodiment of the invention encompasses the use of sucrose octaacetate alone or in conjunction with the bittering agent denatonium benzoate.

Another embodiment of the invention encompasses a method of use for a flexible wound dressing, wherein the flexible wound dressing further comprises a pharmacological, healing or antibiotic agent such as emollients and lipid layer enhancers to aid in wound healing by preventing the drying action of the alcohol carriers. Examples are glycerin and lipid layer enhancer surfactants.

Another embodiment of the invention encompasses a method of use for a flexible wound dressing, wherein the flexible wound dressing further comprises a pharmacological or healing agent such as allantoin as a vulnerary and debriding agent as an aid in wound healing.

Another embodiment of the invention encompasses a method of use for a flexible wound dressing, wherein the flexible wound dressing further comprises a pharmacological, healing or antibiotic agent compounds such as N-Methylpyrrolidone and 2-Pyrrolidone to promote solubility of the above listed ingredients in alcoholic solutions. The preferred range is from 5 to 50% of either compound or combination thereof with a preferred total level of 18%.

Another embodiment of the invention encompasses a method of use for a flexible wound dressing, wherein the flexible wound dressing further comprises a pharmacological, healing or antibiotic agent solvents such as polyvinylpyrrolidone and polypyrrolidone which are used to promote solubility of the constituent ingredients and to modify the barrier attributes of the various combinations of ingredients in the product.

Another embodiment of the invention encompasses a method of use for a flexible wound dressing, wherein the flexible wound dressing further comprises a pharmacological, healing or antibiotic agent solvents such as ethyl alcohol, SDA ethyl alcohol, isopropyl alcohol, n-propyl alcohol, methanol and/or other antibacterial alcohols familiar to those skilled in the art with a preferred use of ethyl alcohol and/or SDA ethyl alcohols, either 190 or 200 proof.

These and other aspects, objects, features and advantages of the present invention will be more clearly understood and appreciated from a review of the following detailed description of the preferred embodiments and appended claims.

### BRIEF DESCRIPTION OF THE FIGURES

FIG. 1 shows the percent of preweaning mortality by age with trend lines and total preweaning mortality.

FIG. 2 shows improved activity in BARRIER® Treatment Piglets.

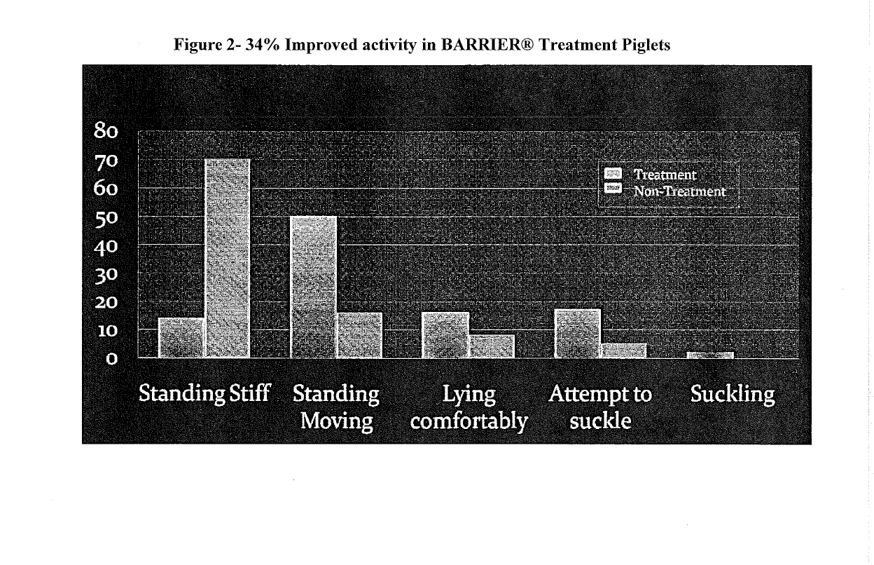

FIG. 3 shows reduction in percent of pigs that bled in BARRIER® Treatment group.

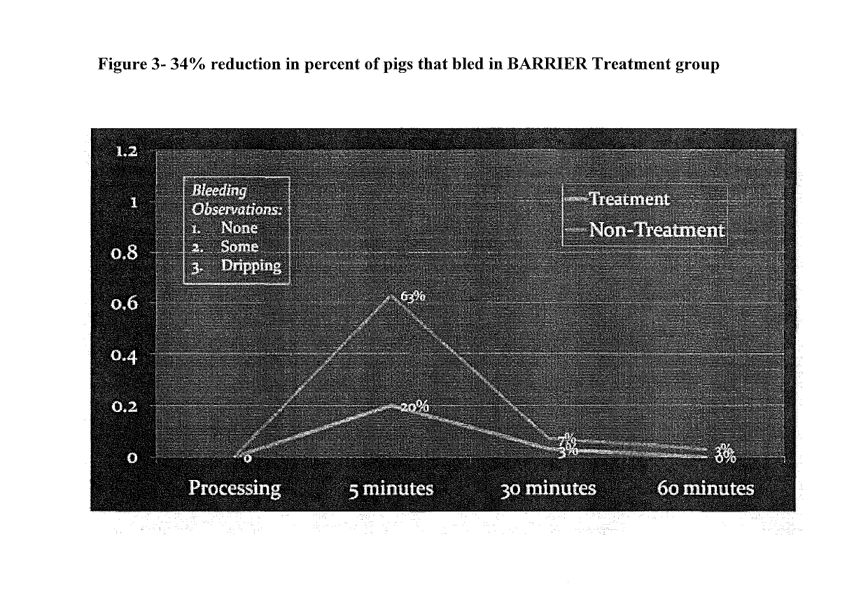

FIG. 4 shows Percent Inflammation Post Processing at day 1, 2 and 3—26% of Treatment group healed faster.

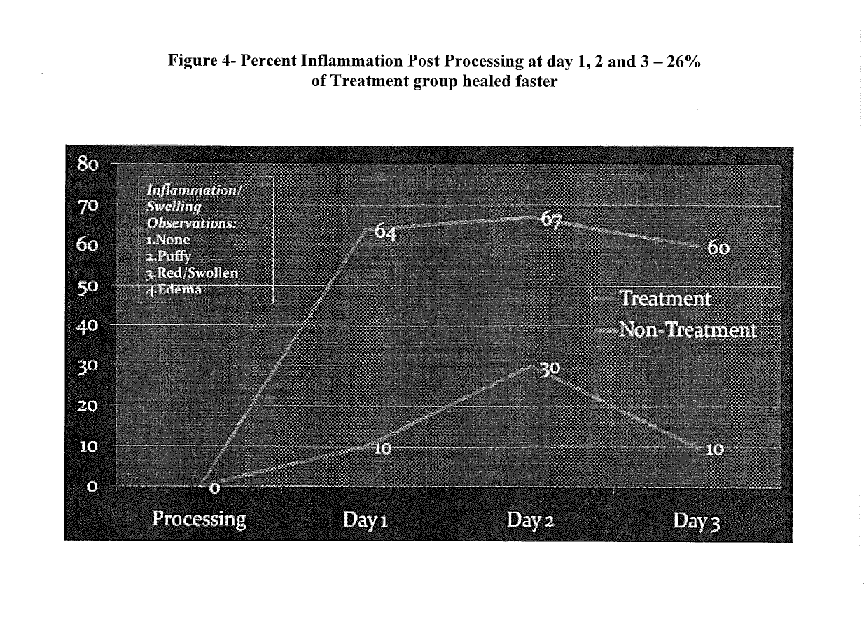

FIG. 5 shows the number of piglets with an abscess post processing at Day 1, 2 and 3.

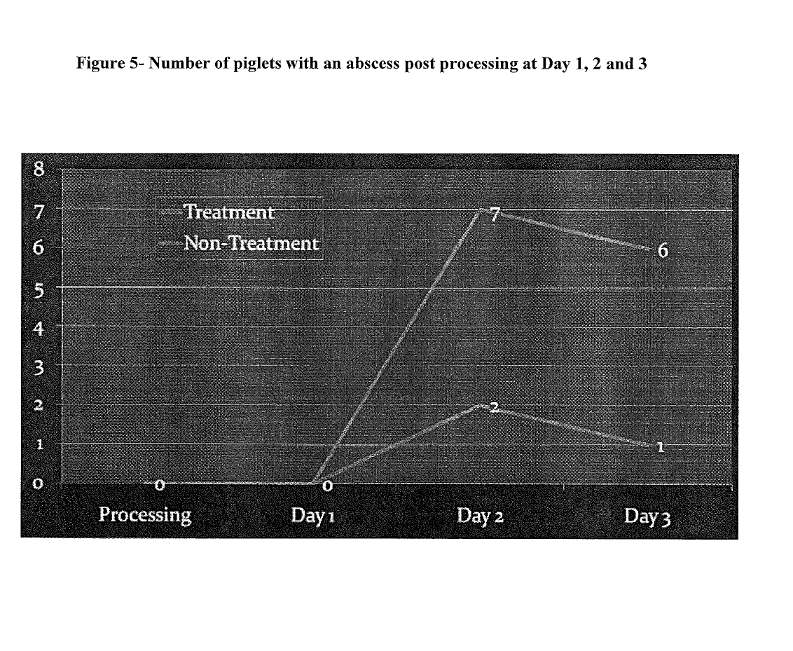

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implicated in other compositions and methods, and that any such variation would be within such modifications that do not part from the scope of the present invention. Before explaining the disclosed embodiments of the present invention in detail, it is to be understood that the invention is not limited in its application to the details of any particular embodiment shown, since of course the invention is capable of other embodiments. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

The present invention contemplates a waterless preparation which quickly kills bacteria on the skin at a wound or surgical site; a long acting (days) bacterial killing action; a product that dries quickly to form a flexible barrier over the skin which is durable that can last up to 3-4 days; a durable barrier that can be washed off with water with some effort; a stable formulation which is stable for up to 3 years; and a broad spectrum of activity against common bacteria and viruses found on the skin.

Additional attributes may be added individually or in combination to the preparation including:

A. The amine type topical anesthetic lidocaine (base or ionic forms such as HCl) for rapid pain relief up to 5% concentration(Prilocaine and other related amine compounds will also work) with a preferred use level of 2%.

B. The ester type topical anesthetic Benzocaine for rapid pain relief up to 5% concentration (Tetracaine and other ester type compounds will also work) with a preferred use level of 2%.

C. Mixtures of amine type topical anesthetics such as the popular EMLA which is a mixture of 2.5% Lidocaine and 2.5% prilocaine.

D. Mixtures of ester type topical anesthetics.

E. Mixtures of amine type and ester type topical anesthetics.

F. Pramoxine HCl for longer duration pain relief and antipruritic activity

G. Permethrin both natural and artificial as insect repellant

H. Citronella as a natural insect repellant, bittering agent and olfactory repellent.

I. Denatonium benzoate as a bittering agent alone or in combination with sucrose octaacetate.

J. Sucrose octaacetate as a bittering agent alone or in combination with denatonium benzoate.

K. Emollients for skin integrity and to provide increased flexibility such as glycerin, aloe vera, lipid layer enhancer surfactants, etc.

L. Asaminoglycoside antibiotics, cephalosporins, carbapenems, quinolone (fluoroquinolone), macrolide antibiotics, penicillins, sulfonamides, tetracyclines, oxazolidinones, lipopeptides, gemifloxacin, ketolides, clindamycin, metronidazole, vancomycin, rifabutin, rifampin, nitrofurantoin, chloramphenicol.

The compositions and methods of the present invention are useful in a wide variety of applications. For example, and not limiting in any way, the present invention may generally be used as a:

1. Flexible barrier sprayed over the top of stitches post operatively;

2. Flexible barrier for use as a teat dip to prevent udder infections;

3. Post trauma spray for wounds as a field treatment to reduce wound contamination and pain;

4. A flexible barrier when sprayed on the hands that acts like a disinfecting glove-continuously disinfecting over hours or days without an allergic or immune response;

5. Can be sprayed on both skin and hair forming a coating that isolates the contamination from the wound. Effective on traumatic wounds for reducing bacterial contamination for an extended period of hours to days;

6. Military use for treating field wounds and providing pain relief and preventing insect attraction to the wound.

Particular applications of the present invention also include, but are not limited to:

1. Topically as a pre-surgical preparation;

2. Topically on castration, tail docking, dehorning and branding wounds in animals to control pain and infection;

3. Topically post surgically to prevent chewing and biting, pain and infection in companion animals and livestock;

4. Topically as a long lasting livestock teat dip;

5. Topical treatment for minor skin infections with pain and itch relief;

6. Topical treatment for lick granulomas in dogs;

7. Topical treatment for topical Staphylococcal infections in pets and humans with pain and itch relief and anti-chewing/licking in animals;

8. Long acting treatment for bed sores with pain relief;

9. Topical treatment for birds for treatment and prevention of pecking.

One particular embodiment of the present invention provides a wound care spray with pain relief. The wound care spray may comprise 2% titratable iodine that kills up to 99% of surface germs that may cause an infection. The wound care spray may also include lidocaine to alleviate pain in wounds and surgical incisions. Wound care sprays in accordance with this embodiment of the present invention may have a natural anti-biting/chewing agent to help prevent self-mutilation and/or cohort licking, biting and chewing. Wound care sprays in accordance with this embodiment of the present invention typically dry in approximately 60 seconds and form a highly visible and durable coating. Wound care sprays in accordance with this embodiment of the present invention may be packaged in bulk gallons and a 16 oz. bottle that comes with a trigger sprayer. To apply, hold the sprayer about 4-6 inches from the area to be treated and sprayed one or more times.

### EXAMPLE 1

### BARRIER® Waterless Surgical Prep 2% Available Iodine

Active ingredients:

Povidone-Iodine (Antiseptic)

(2% available iodine)

Ethyl Alcohol 80.6% v/v (Antiseptic)

Isopropyl Alcohol 4.0% v/v (Antiseptic)

For use in cattle, sheep and swine as an aid in reducing surface bacteria. BARRIER® Waterless Surgical Prep provides rapid antimicrobial kill of a broad spectrum of microorganisms, including antibiotic-resistant strains, with greater than 99% microbial kill in 15 seconds or less. Waterless Surgical Prep contains 2% titratable iodine that kills up to 99% of surface germs that may cause an infection. The composition dries quickly and forms a visible and durable coating and allows quick and effective surgical site preparation. The composition has the same effect on reducing bacteria at the surgical site as conventional iodine scrub with alcohol rinse procedures. Yet the site remains dry. provides continuous disinfectant activity and a physical barrier on the skin and hair The composition may be used to clean the area of wounds that are too difficult to clip dramatically reduces surgical prep time.

The 16 oz. bottle is packaged with a trigger sprayer. To apply, hold sprayer about 4-6 inches from area to be treated and spray one or more times. Repeat as needed. Forms a highly visible coating that is durable.

TABLE 1
BARRIER ® Waterless Surgical Prep Clinical Comparison

| | Average | Average after | Average after |
|---|---|---|---|
| | all skin | 5 minutes | 60 minutes |
| BARRIER ® | (col./ml) | (col/ml) | (col/ml) |
| BARRIER ® Waterless | 628 | 57 | 20 |
| surgical Prep | | | |
| Conventional scrub and | 628 | 67 | 187 |
| alcohol rinse | | | |

Standard plate count - colonies/ml by treatment and time

### EXAMPLE 2

### BARRIER® Livestock Wound Care

Active ingredients:

Povidone-Iodine, 2% available iodine, (Antiseptic)

Bitrex™ Denatonium Benzoate (Bitter Agent)

Ethyl Alcohol 80.6% v/v (Antiseptic)

Isopropyl Alcohol 4.0% v/v (Antiseptic)

For use in cattle, sheep and swine. For use in animals as an aid in reducing chewing on wounds and surgical incisions. BARRIER® Livestock Wound Care, ingredients identified above, is a 2% titratable iodine that kills up to 99% of surface germs that may cause an infection. BARRIER® Livestock Wound Care has an anti-biting/chewing agent to help prevent self-mutilation and/or cohort licking, biting and chewing and dries in approximately 60 seconds and forms a highly visible and durable coating.

The 16 oz. bottle comes with a trigger sprayer. Hold sprayer about 4-6 inches from area to be treated. Spray one or more times, allow to dry. Repeat as needed. Forms a highly visible coating that is durable.

### Wound Care Spray Product Comparison

| | BARRIER ® | | | |
|---|---|---|---|---|
| | Livestock | | Iodine 1% | |
| Features | Wound Care | Triodine-7 | Solution | Stop Bite |
| Labeled for | YES | YES | NO | YES |
| Animals | | | | |
| Instant Kill <10 | YES | NO | YES | NO |
| seconds | | | | |
| Prolonged Kill >10 | YES | YES | NO | NO |
| 10 seconds | | | | |
| Anti-Chew/Anti- | YES | NO | NO | YES |
| Bite | | | | |
| Non-Staining | YES | NO | NO | YES |
| Scented | YES | NO | NO | NO |
| Fast Drying | YES | NO | NO | NO |
| Washes Off Hands | YES | YES | NO | NO |
| Visible | YES | YES | YES | YES |
| Residual Activity | YES | MAYBE | NO | NO |

### EXAMPLE 3

### BARRIER® Wound Care Spray with Pain Relief 2% Available Iodine with Lidocaine

Active ingredients:

Povidone-Iodine, 2% available iodine, (Antiseptic)

Bitrex™ Denatonium Benzoate (Bitter Agent)

Lidocaine 2% w/v (Pain Relief)

Ethyl Alcohol—less than 80% (Antiseptic)

Isopropyl Alcohol—less than 5% (Antiseptic)

For topical use on animals as an aid in reducing pain, licking and chewing on wounds and surgical incisions. Kills up to 99% of surface germs in 15 seconds or less that can potentially cause an infection. BARRIER® Wound Care Spray with Pain Relief, ingredients identified above, is a 2% titratable iodine that kills up to 99% of surface germs that may cause an infection. BARRIER® Wound Care Spray contains lidocaine to alleviate pain in wounds and surgical incisions. BARRIER® Wound Care Spray has a natural anti-biting/chewing agent to help prevent self-mutilation and/or cohort licking, biting and chewing and dries in approximately 60 seconds and forms a highly visible and durable coating.

The 16 oz. bottle comes with a trigger sprayer; pour gallon into a spray bottle dialed to spray pattern. Hold sprayer about 4-6 inches from area to be treated. Spray one or more times, allow to dry. Repeat as needed. Forms a highly visible coating that is durable.

### Wound Care Spray Product Comparison

| | BARRIER II | Original | | |
|---|---|---|---|---|
| | Livestock | BARRIER ® | | Iodine |
| | Wound | Wound | | 1% |
| Features | Care | Care Spray | Triodine-7 | Solution |
| Labeled for | YES | YES | YES | NO |
| Animals | | | | |
| Instant Kill <10 | YES | YES | NO | YES |
| seconds | | | | |
| Prolonged Kill >10 | YES | YES | YES | NO |
| seconds | | | | |
| Anti-Chew/Anti- | NO | YES | NO | NO |
| Bite | | | | |
| Non-Staining | YES | YES | NO | NO |
| Scented | YES | YES | NO | NO |
| Fast Drying | YES | YES | NO | NO |
| Washes Off Hands | YES | YES | YES | NO |
| Visible | YES | YES | YES | YES |
| Residual Activity | YES | YES | MAYBE | NO |

### EXAMPLE 4

Additional information on the stability of the formulation. This is accelerated data on a production batch, there is similar data showing at least 2 years of stability to date.

First production batch of Waterless Surgical Prep 035001C, 60 days stability (R&D program) samples tested:

Available Iodine is 2.3% w/v—complies with Specs (1.7-2.4%)

Effect of application to hands of PVPI Waterless surgical prep spray verses traditional hand scrub with Betadine surgical scrub and water.

| | T-0 | T-5 | T-65 |
|---|---|---|---|
| PVPI Waterless | 367 | 19 | 5 |
| Traditional Scrub | 369 | 20 | 137 |

These represent colony forming units post swab after wearing gloves for 60 minutes. Gloves were applied at 5 minutes after completing the surgical scrub. This is an indication of duration of effect verses traditional standard methods used today. Appears to be that as long as the Waterless PVPI is coating the hand there is a positive effect of reducing skin bacteria. The product has been shown to persist experimentally for over 12 hours without losing integrity when covered by a surgical glove. It offers an effective barrier to bacterial contamination in the event of glove failure. It also provides a similar effect on reducing contamination from the skin around and in a wound environment which experimentally will last up to 24 hours after a single application. It can be washed off with water with moderate physical action. Once dried it is resistant to moisture and will not wash off readily with a stream of water or the action of bleeding.

It provides an effective long term physical and chemical barrier to re-colonization of the skin following application unlike traditional methods of scrubbing or water based antibacterial formulations.

It also prevents the need to physically interact with the wound or skin around a surgical site reducing the potential for additional physical microbiological cross contamination.

This same effect can be applied to inanimate objects also. It can be sprayed on instruments and allowed to dry to provide a bacteria free environment in a field setting and to reduce the potential for recontamination if working in contaminated wound setting

### EXAMPLE 5

### Study of BARRIER on Mortality Rate After Castration and Tail Docking

An initial study was completed in February 2011 on piglets looking at the effect BARRIER® has on castration and tail docking in the farrowing crate. A summary of what we found verses controls are:

1. Reduced Bleeding in the Barrier II treated piglets five minutes post application

2. Improved activity at five and thirty minutes post application

3. Eighty five percent less infections seven days post treatment

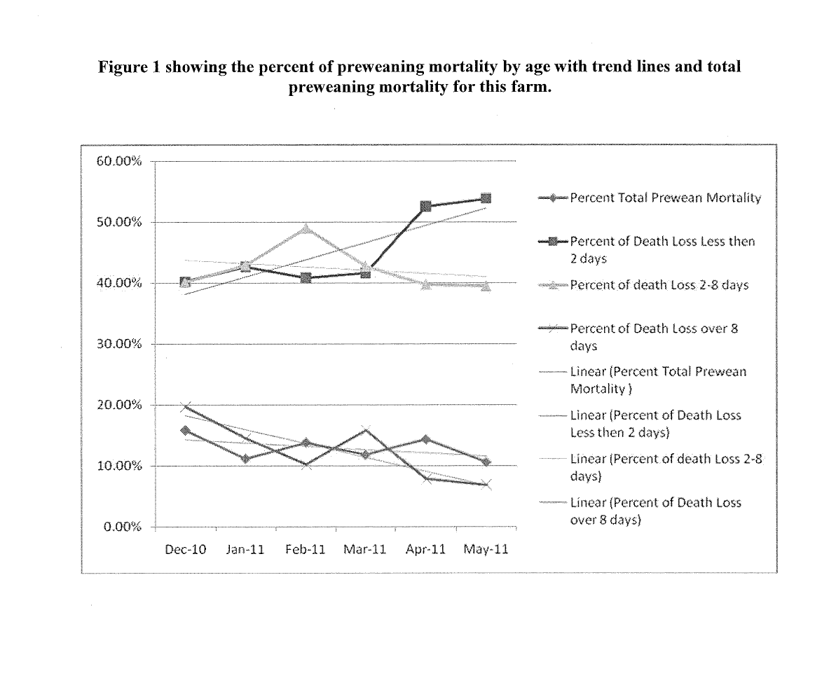

Following the completion of the study, the study site has continued to purchase and use Barrier II routinely for castration and tail docking and their Pig Champ records are showing a marked reduction in preweaning mortality in day 8 and older piglets in the magnitude of five additional full value piglets at weaning per thousand pigs treated with Barrier II. Most of this benefit related to less laid on piglets after 8 days of age most likely due to the reduction in post surgical infection rates that we saw in our initial study. See the Chart in FIG. 1 showing the percent of preweaning mortality by age with trend lines and total preweaning mortality for this farm.

Barrier with pain Relief started being used the second week of February 2011. Prior to using Barrier, this farm was routinely using 0.5 percent tincture of iodine. We believe based on this data, Barrier II—Povidone Iodine wound spray with Pain Relief—is a good way to lower preweaning mortality and address the needs of the piglet following these routine surgical procedures.

### EXAMPLE 6

### BARRIER® Piglet Study Design

46 crates

Processing procedure
Excede® shot given to all piglets at this time
Castration and tail dock males
Females are tail docked day of farrowing

Randomization
Individual pig blocked by litter/crate

Treatments
BARRIER® treatment—tail and castration site (127 piglets)
Non-treatment—(119 piglets)
   BARRIER Treatment and Non-treatment groups were the same quality prior to treatment

BARRIER® Treatment piglets vs. Non-treatment
34% Improved activity in treated piglets over 60 minutes
32% Reduced bleeding over 60 minutes
26% Reduced in average inflammation over 72 hour period
23% Reduction in abscessation over 72 hour period

TABLE 1
Aurora Pharmaceutical, LLC Barrier Wound Care Spray with Pain Relief & Proxamine HCl

2-Dec-10

| 1 | | | | 2 | | | | 3 | | | | 4 | | | | 5 | | | |
|---|---|---|---|---|---|---|---|---|---|---|---|---|---|---|---|---|---|---|---|
| Glycerin | | | | Croda PE/F 87 Flakes | | | | Croda PE/F 108 Flakes | | | | Croda PE/F 127 Flakes | | | | Plasdone K-29/32 | | | |
| PVPI 20% | | | | PVPI 20% | | | | PVPI 20% | | | | PVPI 20% | | | | PVPI 20% | | | |
| Amt/ | | Amt/ | | Amt/ | | Amt/ | | Amt/ | | Amt/ | | Amt/ | | Amt/ | | Amt/ | | Amt/ | |
| 100 mL | | 500 mL | | 100 mL | | 500 mL | | 100 mL | | 500 mL | | 100 mL | | 500 mL | | 100 mL | | 500 mL | |
| 20 | g | 100 | g | 20 | g | 100 | g | 20 | g | 100 | g | 20 | g | 100 | g | 20 | g | 100 | g |
| 55 | g | 275 | g | 55 | g | 275 | g | 55 | g | 275 | g | 55 | g | 275 | g | 55 | g | 275 | g |
| (300 | mg) | (1500 | mg) | (300 | mg) | (1500 | mg) | (300 | mg) | (1500 | mg) | (300 | mg) | (1500 | mg) | (300 | mg) | (1500 | mg) |
| 1.272 | g | 6.36 | g | 1.272 | g | 6.36 | g | 1.272 | g | 6.36 | g | 1.272 | g | 6.36 | g | 1.272 | g | 6.36 | g |
| 1 | g | 5 | g | 1 | g | 5 | g | 1 | g | 5 | g | 1 | g | 5 | g | 1 | g | 5 | g |
| 2 | g | 10 | g | 2 | g | 10 | g | 2 | g | 10 | g | 2 | g | 10 | g | 2 | g | 10 | g |
| 0.5 | g | 2.5 | g | 0.5 | g | 2.5 | g | 0.5 | g | 2.5 | g | 0.5 | g | 2.5 | g | 0.5 | g | 2.5 | g |
| 1 | g | 5 | g | 1 | g | 5 | g | 1 | g | 5 | g | 1 | g | 5 | g | 1 | g | 5 | g |
| 2 | g | 10 | g | | | | | | | | | | | | | | | | |
| | | | | 2 | g | 10 | g | | | | | | | | | | | | |
| | | | | | | | | 2 | g | 10 | g | | | | | | | | |
| | | | | | | | | | | | | 2 | g | 10 | g | | | | |
| | | | | | | | | | | | | | | | | 2 | g | 10 | g |
| 100 | mL | 500 | mL | 100 | mL | 500 | mL | 100 | mL | 500 | mL | 100 | mL | 500 | mL | 100 | mL | 500 | mL |

TABLE 2
Barrier Wound
Care Spray with
Pain Relief and
Fly Control

| | 1 | | | | 2 | | | |
|---|---|---|---|---|---|---|---|---|
| | PVPI 20% | | | | PVPI 20% | | | |
| | Amt/ | | Amt/ | | Amt/ | | Amt/ | |
| Ingredient | 100 mL | | 500 mL | | 100 mL | | 500 ml | |
| Povidone Iodine 10% Available | 20 | g | 100 | g | 20 | g | 100 | g |
| Iodine 30/06 BASF | | | | | | | | |
| Ethyl Alcohol 3-C 190 Proof | 60 | g | 300 | g | 60 | g | 300 | g |
| Specific Gravity 0.807-0.811 | | | | | | | | |
| Bitrex Denatonium Benzoate | (300 | mg) | (1500 | mg) | (300 | mg) | (1500 | mg) |
| 250 mg/mL in PG. d = 1.06 g/ml | 1.272 | g | 6.36 | g | 1.272 | g | 6.36 | g |
| Sucrose Octaacetate | 1 | g | 5 | g | 1 | g | 5 | g |
| Lidocaine | 2 | g | 10 | g | 2 | g | 10 | g |
| Citronella Oil - Java Type 32-45% | 0.2 | g | 1.0 | g | | | | |
| Citronellal | | | | | 0.2 | g | 1.0 | g |
| Piperonyl Butoxide | 1 | g | 5 | g | 1 | g | 5 | g |
| Pyrethrins | 0.1 | g | 0.5 | g | 0.1 | g | 0.5 | g |
| Aloe Vera | 0.1 | g | 0.5 | g | 0.1 | g | 0.5 | g |
| Glycerin | 2 | g | 10 | g | | | | |
| Plasdone K-29/32 ISP Linear | | | | | 2 | g | 10 | g |
| Homopolymer of Vinylpyrrolidone | | | | | | | | |
| q.s./Ethyl Alcohol 3-C 190 Proof | 100 | mL | 500 | mL | 100 | mL | 500 | mL |

While the invention has been described with reference to certain exemplary embodiments thereof, those skilled in the art may make various modifications to the described embodiments of the invention without departing from the scope of the invention. The terms and descriptions used herein are set forth by way of illustration only and are not meant as limitations. In particular, although the present invention has been described by way of examples, a variety of compositions and methods would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A method for treating a wound comprising
(a) applying a topical organic iodine complex alcohol solution to the skin of a human or animal; and
(b) allowing a durable barrier to faun, wherein the durable barrier lasts for 24 hours or more;
wherein the durable barrier provides antibacterial, antifungal, and antiviral activity.

2. The method of claim 1 whereby the organic iodine complex is Povidone Iodine (PVPI).

3. The method of claim 1 whereby the organic iodine complex alcohol solution contains 0.05 to 5.0 percent titratable iodine with a preferred concentration of 2.0 percent titratable iodine.

4. The method of claim 1 whereby the organic iodine complex alcohol solution is Nonylphenoxy-polyethyoxyethanol-iodine complex (CAS 35860-86-7).

5. The method of claim 4 whereby the organic iodine complex alcohol solution contains 0.05 to 5.0 percent titratable iodine with a preferred concentration of 1.0 percent titratable iodine.

6. The method of claim 1 whereby the organic iodine complex alcohol solution is a mixture of two iodine complexes with a range of 0.05 to 5.0 percent titratable iodine from PVPI and a range of 5.0 to 0.05 percent titratable iodine from nonylphenoxypolyethyoxyethanol-iodine complex.

7. The method of claim 1 whereby the organic iodine complex alcohol solution comprises a topical anesthetic.

8. The method of claim 7 whereby the topical anesthetic is pramoxine HCl.

9. The method of claim 1 whereby the topical organic iodine complex alcohol solution comprises permethrin.

10. The method of claim 1 whereby the topical organic iodine complex alcohol solution comprises citronella.

11. The method of claim 1 whereby the topical organic iodine complex alcohol solution comprises denatonium benzoate.

12. The method of claim 11 whereby the topical organic iodine complex alcohol solution comprises sucrose octaacetate.

13. The method of claim 1 whereby the topical organic iodine complex alcohol solution further comprises emollients and lipid layer enhancers.

14. The method of claim 1 whereby the topical organic iodine complex alcohol solution further comprises allantoin.

15. The method of claim 1 whereby the topical organic iodine complex alcohol solution further comprises N-Methylpyrrolidone or 2-Pyrrolidone.

16. The method of claim 1 whereby the topical organic iodine complex alcohol solution further comprises polyvinylpyrrolidone or polypyrrolidone.

17. A method for treating surgical incisions after castration or tail docking comprising
(a) applying a topical organic iodine complex alcohol solution to the skin of an animal; and
(b) allowing a durable barrier to form, wherein the durable barrier lasts for 24 hours or more;
wherein the durable barrier provides antibacterial, antifungal, and antiviral activity.

18. The method of claim 17 whereby the organic iodine complex is Povidone Iodine (PVPI).

19. A method for enhancing the normal healing process of a wound comprising
(a) providing a flexible wound dressing comprising a topical organic iodine complex alcohol solution to the skin around the wound;
(b) allowing the flexible wound dressing to form durable barrier;
wherein the flexible wound dressing lasts for 24 hours or more and provides antibacterial, antifungal and antiviral activity, and wherein the flexible wound dressing comprises an organic iodine complex alcohol solution.

20. The method of claim 19, wherein the organic iodine complex is Povidone Iodine (PVPI).
